(19) United States
(12) Patent Application Publication
(10) Pub. No.: US 2001/0000795 A1
(43) Pub. Date: May 3, 2001

(54) Leakage prevention means in a material for distributing fluid and method of making same

(76) Inventors: Charles Edward Bolian II, Buford, GA (US); Michael David Powers, Canton, GA (US)

Correspondence Address: James B. Robinson, Kimberly-Clark Worldwide, Inc., 401 North Lake Street, Neenah, WI, 54957-0349, US

(21) Appl. No.: 09/737,875

(22) Filed: Dec. 15, 2000

Related U.S. Application Data
(62) Division of application No. 09/329,414, filed on Jun. 10, 1999.

(51) Int. Cl. A61F 13/15; B05D 5/00

(52) U.S. Cl. 604/381; 604/358; 602/41; 427/256

(57) ABSTRACT

The present invention provides a material for distributing liquid and a method for making such a material. The material includes a substrate permeable to a liquid and a chemical treatment applied to at least a portion of the substrate. The substrate further includes a leakage prevention means of at least one area of an increased concentration of the chemical treatment such that the chemical treatment is non-uniform along a planar dimension of the substrate and creates an area of preferential flow through the substrate. The chemical treatment useful for application to the substrate preferably induces wetting of the substrate by a liquid. The material for distributing liquid has best utility as a component of an absorbent article, e.g. a liner of a diaper. In a preferred embodiment, the substrate is attached to an absorbent layer such that the absorbent layer receives and retains the liquid when the liquid flows from a first planar dimension to a second planar dimension. Preferably, the absorbent layer is attached to an inner face of the substrate in face-to-face juxtaposition. The leakage prevention means is generally positioned in the absorbent article to prevent leakage out of the periphery of said absorbent article and the periphery is generally less wettable than the leakage prevention means.

### FIELD OF THE INVENTION

1. The present invention is directed to a material for distributing liquid and a method for making such a material, such that the material includes a substrate permeable to a liquid and a chemical treatment applied to at least a portion of the substrate. A leakage prevention means is formed on the substrate of at least one area of an increased concentration of the chemical treatment such that the chemical treatment is non-uniform along a planar dimension of the substrate and creates an area of preferential flow through the substrate.

### BACKGROUND OF THE INVENTION

2. Substrates such as nonwoven materials are known in the art and are used especially as components of absorbent articles and disposable items because of the relatively low cost of such materials. As used herein, the term “absorbent articles” refers to devices which absorb and contain liquids, fluids and exudates. More specifically, absorbent articles refer to devices that are placed against or in proximity to the body of the wearer to absorb and contain the various fluids discharged from the body, and is intended to include diapers, training pants, absorbent underpants, incontinence products, feminine hygiene products, medical applications such as surgical drapes, gowns, facemasks, and bandages, articles of clothing or portions thereof including workwear and lab coats, and the like. The term “disposable” is used herein to describe absorbent articles not intended to be laundered or otherwise restored or reused as an absorbent article.

3. There remains in practice, though, a need for such a substrate, which when in use, serves as a barrier to leaking of such liquids. One area that continues to receive much attention and research in the technical community is the prevention of leaking of liquids. A substrate useful in prevention of leaking, as for example a component of a diaper, includes leg flaps and waistbands, which typically perform the same functions, that is, to improve the fit (and comfort) of the article and to prevent leaking. A typical leg flap usually includes a gathering of material to “cinch-up” the article, such as a diaper, around the leg of a wearer. As used herein, the term “leg flap” would be understood by those skilled in the art of disposable personal care articles such as diapers to mean that portion of the diaper which is mechanically gathered around the leg of the wearer to prevent leaking of such a fluid or liquid, e.g. urine. Unfortunately, such devices have proven to be unreliable as the only means of capturing these fluids.

4. Also known in the art are various methods of applying a coating of, for instance a wetting agent, to wick away exudates from the surface of the skin to assist in avoidance of irritation to the skin. Specifically, U.S. Pat. No. 5,792,404 to Cree et al., assigned to The Procter & Gamble Company of Cincinnati, Ohio, describes a complicated approach for creating a “plurality of surface energy gradients . . . to exert a force on a fluid” to effect the transport of fluids away from the surface of the material, and hence, the user. Such uses of wetting agents have not, to-date, been used to assist in prevention of leaking of the material. One theory is that these agents may, in fact, contribute to leaking of the garment since these agents are used for skin wellness purposes to wick the fluid away from the skin and ultimately to the outer periphery of the garment. When such wetting agents, the leg flap is usually under an even greater strain to contain the fluids.

5. Also known in the art are various processes for applying chemical treatments to substrates and the like. One common feature of these prior art processes is the attempt to achieve a uniform application of the agent. In fact, the literature is replete with attempts to avoid the non-uniform application of coating agents. For instance, there are references that provide a means for preventing the accumulation of coating on the corners (or edges) of an applicator roll to provide a uniform distribution of coating on the surface of the material.

6. The article and method of the present invention overcomes these prior shortfalls and takes advantage of the accumulation of an applied chemical treatment in such a way as to form a leakage prevention means designed to keep the article from leaking.

### SUMMARY OF THE INVENTION

7. The present invention provides a material for distributing liquid and a method for making such a material. The material includes a substrate permeable to a liquid and a chemical treatment applied to at least a portion of the substrate. The substrate further includes a leakage prevention means of at least one area of an increased concentration of the chemical treatment such that the chemical treatment is non-uniform along a planar dimension of the substrate and creates an area of preferential flow through the substrate. The material for distributing liquid has best utility as a component of an absorbent article, e.g. a liner of a diaper. In a preferred embodiment, the substrate is attached to an absorbent layer such that the absorbent layer receives and retains the liquid when the liquid flows from a first planar dimension to a second planar dimension. Preferably, the absorbent layer is attached to an inner face of the substrate in face-to-face juxtaposition. The leakage prevention means is preferably positioned in the absorbent article to prevent leakage out of the periphery of said absorbent article.

8. The chemical treatment useful for application to the substrate preferably induces wetting of the substrate by a liquid. As such, the leakage prevention means is generally positioned such that the outer periphery of the substrate is less wettable than the leakage prevention means. The substrate can be made from various materials including a woven fabric, nonwoven fabric, foam, knitted fabric, and film. The material is preferably used in an absorbent article such as a diaper, training pant, absorbent underpant, incontinence product, feminine hygiene products, medical application such as a surgical drape, gown, facemask, and bandage, an article of clothing or portion thereof including workwear and lab coat, and the like.

### BRIEF DESCRIPTION OF THE DRAWINGS

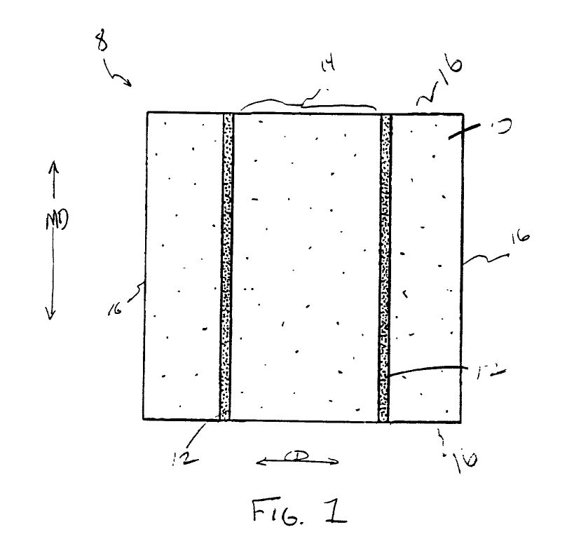

9. FIG. 1 is a top plan view of a material for distributing liquid of the present invention including a leakage prevention means to prevent flow of a fluid out of the periphery (outer edge) of the material.

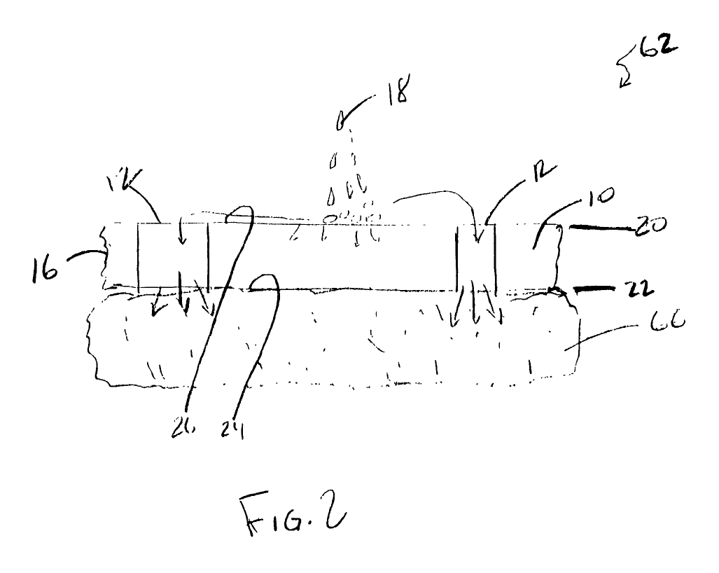

10. FIG. 2 is an enlarged cross-sectional side view of liquid striking the outer surface of a material of the present invention wherein leakage prevention means prevents leakage of the liquid out of the outer periphery of the substrate.

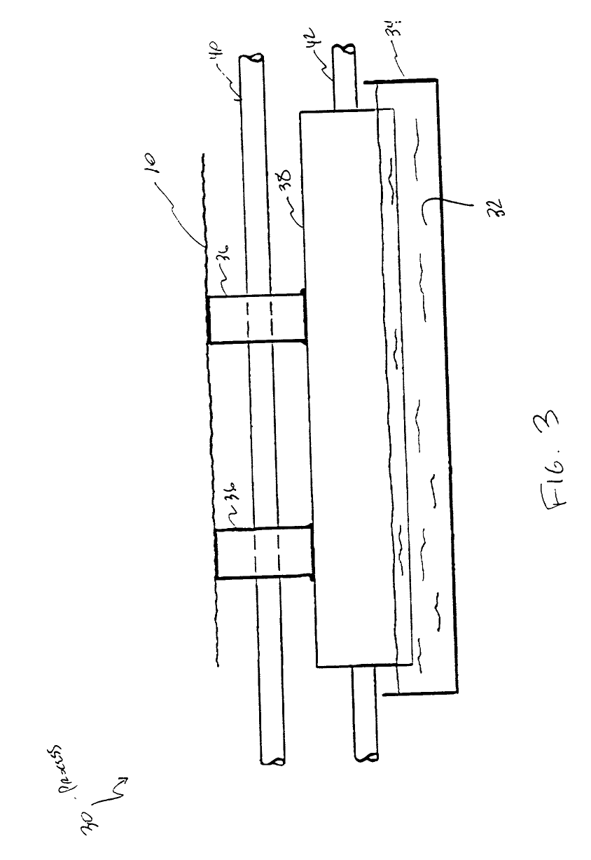

11. FIG. 3 is a schematic drawing of an exemplary process for forming the material for distributing liquid of the present invention.

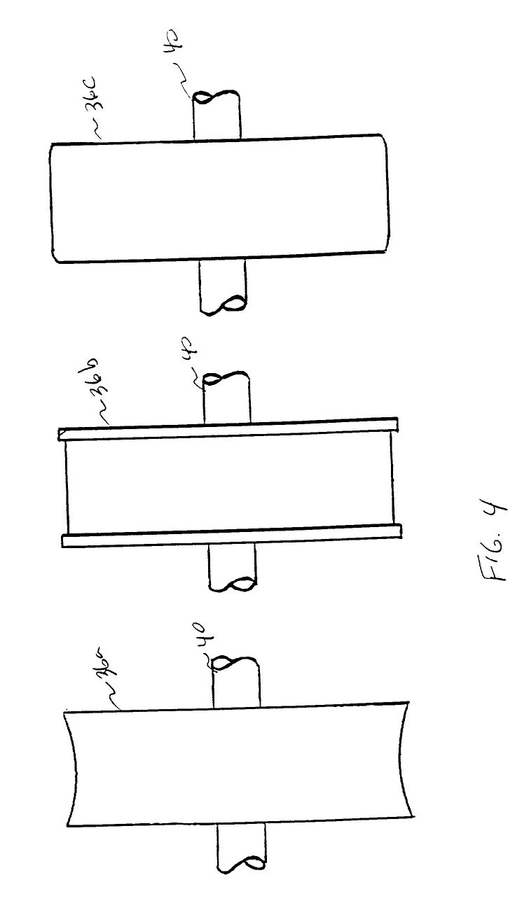

12. FIG. 4 is a side view of exemplary embodiments of the second applicator rolls for applying chemical treatment according to the present invention.

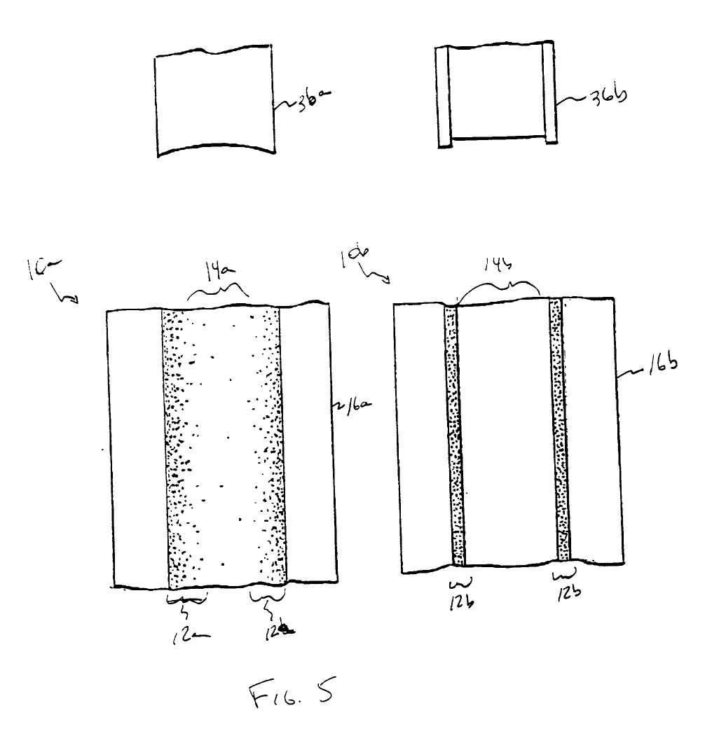

13. FIG. 5 is a cross-sectional side view of exemplary embodiments of the second applicator rolls, along with the imprint these rolls make when applying chemical treatment to the substrate of the present invention.

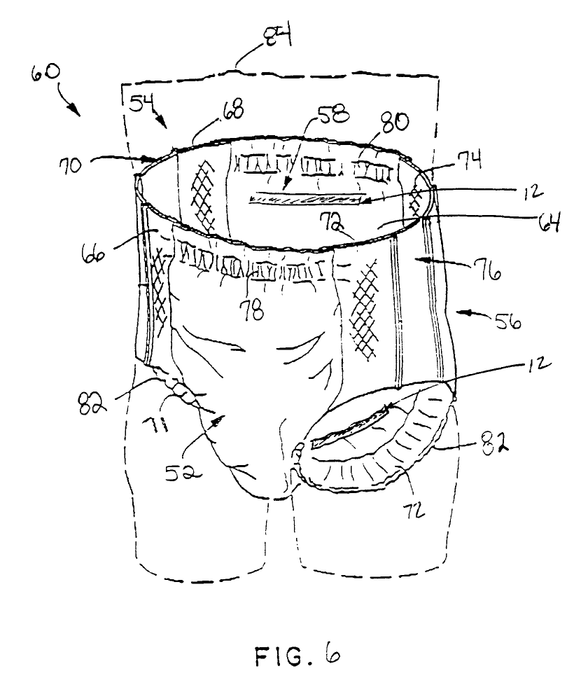

14. FIG. 6 is a perspective view of an exemplary article, in this case, a training pant, utilizing the material for distributing liquid of the present invention as the liner of the garment.

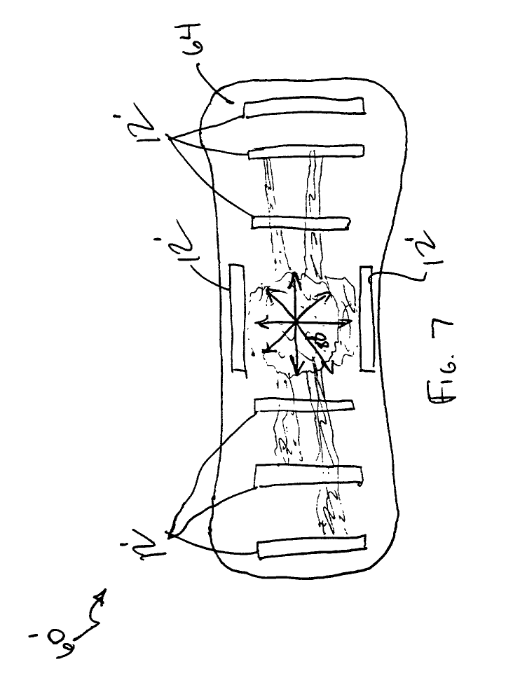

15. FIG. 7 is a top plan view of another article, in this case a sanitary napkin, utilizing the material for distributing liquid of the present invention as the liner of the garment.

### DETAILED DESCRIPTION OF THE INVENTION

16. Referring now to the drawings wherein like reference numerals represent the same or equivalent structure and, in particular, to FIG. 1, the present invention provides a “material for distributing liquid” 8 including a substrate 10 which is permeable to a liquid and a chemical treatment applied to at least a portion of the substrate 10. The substrate has, in this embodiment, four edges of an outer periphery shown at 16, and a center 14 of the substrate between leakage prevention means 12. In FIG. 1, the outer surface of the substrate 10 is shown. The leakage prevention means 12 is an increased concentration of the chemical treatment such that the chemical treatment is non-uniform along a planar dimension of the substrate and creates an area of preferential flow through the substrate. Preferably, the chemical treatment concentration varies from a lower concentration at the center of the substrate to a higher concentration within the treated portion. The leakage prevention means results from a transfer of an accumulation of said chemical treatment on an applicator roll used to apply it to the substrate as will be discussed in more detail below in connection with FIG. 3.

17. As shown in FIG. 1, the leakage prevention means is formed adjacent to the outer periphery 16 along the machine direction of the substrate. It will be understood by one of ordinary skill in the art that such means may also be useful in the cross machine direction. As used herein, the term “machine direction” or MD means the length of a fabric in the direction in which it is produced. The term “cross machine direction” or CD means the width of fabric, i.e. a direction generally perpendicular to the MD.

18. Turning to FIG. 2, an enlarged cross-sectional side view of a preferred embodiment is shown wherein a composite material 62 is formed from substrate 10 which is attached to an absorbent layer 66 such that the absorbent layer 66 receives and retains the liquid 18 when the liquid 18 flows from a first planar dimension 20 to a second planar dimension 22. Preferably, the absorbent layer 66 is attached to an inner face 24 of the substrate 10 in face-to-face juxtaposition. The leakage prevention means 12 is positioned in the composite material 62 to prevent leakage from the outer periphery 16 of the composite material 62. As in most absorbent articles, some of liquid 18 will be absorbed by the substrate 10, and will move through the substrate 10 in the direction of absorbent layer 66. The leakage prevention 12 means formed from at least one area of an increased concentration of the chemical treatment will create an area of preferential flow through the substrate 10 to, for instance, an underlying absorbent layer 66.

19. The location of the leakage prevention means on the substrate will be suitable for the particular intended use. As an example, the material for distributing liquid of the present invention may be used as a liner 64 in, for instance, an absorbent article 60 such as a training pant, as shown in FIG. 6. The absorbent article 60 includes waste containment section 52 and two side panels 54 and 56 defining a waist opening 58 and a pair of leg openings 71 and 72. FIG. 6 illustrates the absorbent article 60 fitted on a wearer's torso 84 shown partially in dashed lines. Side panel 54 includes stretchable side member 76 and stretchable side member 68 connecting intermediate member 70 which is made of a non-stretchable material. Similarly, side panel 56 includes stretchable side member 72 and stretchable side member 74 connecting intermediate member 76 which is made of a non-stretchable material. Absorbent article 60 also includes front waist elastic member 78 and rear waist elastic member 90 for providing additional conformability along waist opening 58. Leg flaps 82 are provided with waste containment section 52 between side panels 54 and 56.

20. The material of the present invention may be used to form various portions of the absorbent article 60 and particularly, the liner 64. In this exemplary article, the leakage prevention means 12 may be placed adjacent to the leg flaps 82, and may also be placed adjacent to the front waist elastic member 78 and rear waist elastic member 90 to assist in the prevention of leakage from the garment. In this embodiment, the leakage prevention means is positioned adjacent to the outer periphery of the liner such that the outer periphery is less wettable than the leakage prevention means.

21. As another example and as shown in FIG. 7, leakage prevention means 12′ are utilized in a liner 64 of an absorbent article 60′, in this case a sanitary napkin. In this instance, it may be useful to provide leakage prevention means 12′ in one or more of the locations shown to prevent leakage of menses along the direction shown by arrows 80.

22. Suitable substrates include woven fabrics, nonwoven fabrics, foams, knitted fabrics, films and combinations of any of the foregoing. If the substrate is a film, the film may be made from either cast or blown processes and will be permeable to liquids. Examples of such a nonwoven web include those formed from a meltblowing process, spunbonding process, coformed process and bonded carded web process.

23. As used herein, the term “nonwoven fabric or web” means a web having a structure of individual fibers or threads which are interlaid, but not in an identifiable manner as in a knitted fabric. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

24. As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns (μm), more particularly, between about 10 and 20 microns (μm).

25. The substrate may also be a meltblown fiber or microfiber layer. The meltblown fiber or microfiber is formed utilizing a conventional meltblowing process. Meltblowing processes generally involve extruding a molten thermoplastic material through a plurality of fine, usually circular, capillaries of a meltblowing die as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., assigned to Exxon Research and Engineering Company. Meltblown fibers are microfibers which are generally smaller than 10 microns (μm) in average diameter, and are generally tacky when deposited onto a collecting surface.

26. As used herein the term “microfibers” means small diameter fibers having an average diameter not greater than about 50 microns (μm), for example, having an average diameter of from about 0.5 microns (μm) to about 20 microns (μm), or more particularly, microfibers may have an average diameter of from about 2 microns (μm) to about 40 microns (μm). Another frequently used expression of fiber diameter is denier, which is defined as grams per 9000 meters of a fiber and may be calculated as fiber diameter in microns (μm) squared, multiplied by the density in grams/cc, multiplied by 0.00707. A lower denier indicates a finer fiber and a higher denier indicates a thicker or heavier fiber. For example, the diameter of a polypropylene fiber given as 15 microns (μm) may be converted to denier by squaring, multiplying the result by 0.89 g/cc and multiplying by 0.00707. Thus, a 15 micron (μm) polypropylene fiber has a denier of about 1.42 (15²×0.89×0.00707=1.415). Outside the United States the unit of measurement is more commonly the “tex”, which is defined as the grams per kilometer of fiber. Tex may be calculated as denier/9.

27. The nonwoven web may be a mixture of various fibers or particulates. For an example of such a mixture, reference is made to U.S. Pat. No. 4,209,563, to Sisson, assigned to the Procter and Gamble Company, incorporated herein by reference, in which elastomeric and non-elastomeric fibers are commingled to form a single coherent web of randomly dispersed fibers. Another example of such a web would be one made by a technique such as disclosed in previously referenced U.S. Pat. No. 4,741,949. That patent discloses a nonwoven material which includes a mixture of meltblown thermoplastic fibers and other materials. The fibers and other materials are combined in the gas stream in which the meltblown fibers are borne so that an intimate entangled commingling of meltblown fibers and other materials, e.g., wood pulp, staple fibers or particulates such as, for example, activated charcoal, clays, starches, or hydrocolloid (hydrogel) particulates commonly referred to as super-absorbent materials occurs prior to collection of the fibers upon a collecting device to form a coherent web of randomly dispersed fibers.

28. The substrate is one which is formed from any material which may be manufactured from suitable thermoplastic polymers or blends containing the same. As used herein, the terms “slayer” or “web” when used in the singular can have the dual meaning of a single element or a plurality of elements. As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the molecule. These configurations include, but are not limited to isotactic, syndiotactic and random symmetries.

29. Useful polymers include polyolefins, for example, polyethylene, polypropylene and polybutene, including ethylene copolymers, propylene copolymers and butene copolymers, including high density polyethylene, low density polyethylene, linear low density polyethylene. A particularly useful polyethylene may be obtained from the U.S.I. Chemical Company under the trade designation Petrothene NA 601 (also referred to herein as PE NA 601 or polyethylene NA 601). Two or more of the polyolefins may be utilized. Other thermoplastic polymers include cellophane, polyvinyl acetate, polyvinyl alcohol, polycaprolactam, polyester, polyamide, polyethylene terephthalate, polybutylene terephthalate, polytetrafluoroethylene, or mixtures or coextrusions of one or more of these materials.

30. The substrates useful in the material of the present invention may additionally be made from elastomeric thermoplastic polymers such as block copolymers including polyurethanes; copolyester elastomers like copolyetheresters; polyamide polyether block copolymers; copolymers of ethylene and at least one vinyl monomer such as, for example, vinyl acetates such as ethylene vinyl acetate (EVA), unsaturated aliphatic monocarboxylic acids, and esters of such monocarboxylic acids; block copolymers having the general formula A—B—A′, A—B or A—B—A—B like copoly(styrene/ethylenebutylene), styrene-poly(ethylene-propylene)-styrene, styrene-poly(ethylene-butylene)styrene, (polystyrene/poly(ethylene-butylene)/polystyrene, poly(styrene/ethylenebutylene/styrene), polystyrene-poly(ethylene-propylene)-polystyrene-poly(ethylenepropylene) and the like. Also, the new class of polymers referred to as single site catalyzed polymers such as “metallocene” polymers produced according to a metallocene process are also useful. For a more detailed description of the metallocene polymers and the process for producing same which are useful in the present invention see commonly assigned PCT Patent Application No. WO 98/29246 to Gwaltney et al., which is incorporated herein by reference in its entirety.

31. The chemical treatment is any chemical treatment which would be useful in forming the leakage prevention means of the present invention. Preferably, the chemical treatment is one that induces wetting of the substrate by the liquid. Examples of such wetting agents include TRITON X102 available from Union Carbide, Ahcovel Base N-62 mixture of sorbitan monooleate and polyethoxylated hydrogenated castor oil, manufactured by ICI (Imperial Chemical Industries), Inc., GLUCOPON 220P available from Henkel, MASIL®SF-19 available from BASF, Inc. and the like.

32. The chemical treatment is made up of from about 1% to about 100% chemical treatment and from about 0% to about 99% water. Preferably, the chemical treatment is made up of about 20% chemical treatment and 80% water.

33. The material for distributing liquid may be used in absorbent articles such as diapers, training pants, absorbent underpants, incontinence products, feminine hygiene products, medical applications such as surgical drapes, gowns, facemasks, and bandages, articles of clothing or portions thereof including workwear and lab coats, and the like.

34. Articles made from the material of the present invention may also have topical treatments applied to them for more specialized functions. Such topical treatments and their methods of application are known in the art and include, for example, alcohol repellency treatments, anti-static treatments and the like, applied by spraying, dipping, etc. An example of such a topical treatment is the application of Zelec® antistat (available from E. I. du Pont de Nemours and Company, Wilmington, Del.).

35. According to the present invention and turning to FIG. 3, a material for distributing liquid may be made by the process of the present invention. The method of applying the chemical treatment to the substrate 10 to form leakage prevention means is accomplished, preferably by providing a first applicator roll 38 which is designed to be partially submersed in a chemical treatment 32 which is contained in a chemical treatment reservoir 34. As the first applicator roll 38 rotates around shaft 42, the chemical treatment 32 is picked up by the roll. At least one, and preferably a plurality of second applicator rolls 36 are positioned on shaft 40 and in contact with the first applicator roll 38, such that during rotation, the chemical treatment 32 is transferred from the first applicator roll 38 to the second applicator rolls 36. The outer face 26 of substrate 10 is positioned in contact with the second applicator rolls 36 such that chemical treatment 32 is thereby transferred to the substrate 10. By applying the chemical treatment 32 in this way, a heavy bead of chemical treatment 32 is formed on the surface of the substrate 10 to form leakage prevention means 12. An additional advantage of applying the chemical treatment as an increased concentration adjacent to the periphery of the substrate is the reduction in exposure of the wearer to the chemical treatment, as applies when the chemical treatment is uniformly applied to the substrate. Preferably, the leakage prevention means 12 is formed at the area of contact between the substrate 10 and an edge of the said second applicator roll 36. It will be understood that the chemical treatment may also be applied to the substrate in varying degrees of concentration between the heavy beads of concentration formed by the edge of the applicator roll, depending upon roll selection. It will further be understood that the second applicator rolls will be sized sufficiently to apply the necessary amount of chemical treatment to the surface of the substrate. As an example, if the substrate were to be utilized as a liner in a diaper, the substrate could be cut into a liner-sized portions for conversion into diapers. The substrate, therefore, may be cut into, for instance, 14 inches (35.14 cm) in the MD and 7 inches (17.57 cm) in the CD. In this case, the applicator roll width may be, for instance 5 inches (12.7 cm) wide which would apply the beads of concentration of chemical treatment along the MD outer periphery, approximately 1 inch (2.54 cm) from the outer periphery. A hold-down or nip roll (not shown) may be placed in contact with the inner face 24 of substrate 10 opposite of the second applicator roll 36 to prevent flapping of the substrate during application of the chemical treatment.

36. Unlike prior methods utilized for application of a chemical treatment to a substrate by roll applications, use of a doctor blade is not helpful in the present invention because accumulation of a concentration of chemical treatment will most likely be removed. Along with the advantageous use of this accumulation, varying degrees of chemical treatment may be applied to the substrate depending on the selection of second applicator roll 36 utilized. Turning to FIGS. 4 and 5, along with a standard roll, the applicator roll may be modified to provide such varying degrees of chemical treatment to the substrate. The second applicator rolls of FIG. 4 include a roll having a concave surface 36a, a roll have grooved ends 36b and a roll having a convex surface 36c.

37. While particular embodiments of the present invention have been illustrated and described, it would be apparent to those skilled in the art that various other changes and modifications can be made without departing from the spirit and scope of the invention. It is therefore intended to cover in the appended claims all such changes and modifications that are within the scope of this invention.

We claim:

1. A material for distributing liquid, comprising:
a) a substrate permeable to a liquid;
b) a chemical treatment applied to at least a portion of said substrate; and
c) leakage prevention means of at least one area of an increased concentration of said chemical treatment, wherein said chemical treatment is non-uniform along a planar dimension of said substrate and creates an area of preferential flow through said substrate.

2. The material of claim 1, wherein said leakage prevention means results from a transfer of an accumulation of said chemical treatment on an applicator to said substrate.

3. The material of claim 1, wherein said chemical treatment further comprises from about 1% to about 100% chemical treatment and from about 0% to about 99% water.

4. The material of claim 1, wherein said chemical treatment induces wetting of said substrate by the liquid.

5. The material of claim 1, wherein said substrate is selected from the group comprising a woven fabric, nonwoven fabric, foam, knitted fabric, and film.

6. The material of claim 5, wherein the material is used in an absorbent article selected from the group comprising a diaper, training pant, absorbent underpant, feminine hygiene product, and incontinence product.

7. The material of claim 5, wherein said material is used in an absorbent article for medical application selected from the group comprising a surgical drape, gown, facemask, and bandage.

8. The material of claim 5, wherein said material is used in an absorbent article selected from the group comprising an article of clothing or portion thereof.

9. The material of any of claims 6, 7 or 8, wherein said leakage prevention means is positioned in said absorbent article to prevent leakage from the periphery of said absorbent article.

10. An absorbent article comprising:
a) a liner permeable to a liquid;
b) a wetting agent applied to at least a portion of said liner; and
c) leakage prevention means of at least one area of an increased concentration of said wetting agent, wherein said wetting agent is non-uniform along a planar dimension of said liner and creates an area of preferential flow through said liner;
wherein said leakage prevention means is positioned in said absorbent article to prevent leakage from the periphery of said absorbent article; and further wherein the periphery of said liner is less wettable than said leakage prevent means.

11. A composite material for distributing and retaining liquid, comprising:
a) a substrate permeable to a liquid;
b) a chemical treatment applied to at least a portion of said substrate;
c) leakage prevention means of at least one area of an increased concentration of said chemical treatment wherein said chemical treatment is non-uniform along a planar dimension of said substrate and creates an area of preferential flow through said substrate; and
d) an absorbent layer in face-to-face juxtaposition with an inner face of said substrate such that it receives and retains the liquid when the liquid flows from a first planar dimension to a second planar dimension of said composite material.

12. The composite material of claim 11, wherein said leakage prevention means results from a transfer of an accumulation of said chemical treatment on an applicator to said substrate.

13. The composite material of claim 11, wherein said chemical treatment further comprises from about 1% to about 100% chemical treatment and from about 0% to about 99% water.

14. The composite material of claim 13, wherein said chemical treatment induces wetting of said substrate by a liquid.

15. The composite material of claim 11, wherein said substrate is selected from the group comprising woven fabric, nonwoven fabric, foam, knitted fabric, and film.

16. The composite material of claim 15, wherein said material is used in an absorbent article selected from the group comprising a diaper, training pant, absorbent underpant, feminine hygiene product, and incontinence product.

17. The composite material of claim 15, wherein said material is used in an absorbent article for medical application selected from the group comprising a surgical drape, gown, facemask, and bandage.

18. The composite material of claim 15, wherein said material is used in an absorbent article selected from the group comprising an article of clothing or portion thereof.

19. The composite material of claim 15, wherein said article of clothing further comprises workwear or a lab coat.

20. The composite material of any of claims 16, 17, 18 or 19, wherein said leakage prevention means is positioned in said absorbent article to prevent leakage from the periphery of said absorbent article.

21. The composite material of claim 20, wherein said leakage prevention means is positioned adjacent to at least one outer edge of said absorbent article.

22. The composite material of claim 21, wherein the periphery of said liner is less wettable than said leakage prevent means.

23. A process for making a material for distributing liquid, comprising:
a) providing a substrate permeable to a liquid;
b) providing an applicator for application of a chemical treatment; and
c) applying said chemical treatment to said substrate to form a leakage prevention means of at least one area of an increased concentration of said chemical treatment, wherein said chemical treatment is non-uniform along a planar dimension of said substrate and creates areas of preferential flow through said substrate to form a material for distributing liquid.

24. The process of claim 23, further comprising:
a) providing a first applicator roll to apply said chemical treatment;
b) applying said chemical treatment to said first applicator roll;
c) contacting said first applicator roll to a second applicator roll, such that said chemical treatment is transferred from said first applicator roll to said second applicator roll; and
d) transferring said chemical treatment to said substrate by contacting said substrate with said second applicator roll.

25. The process of claim 24, further comprising:
a) attaching an absorbent layer in face-to-face juxtaposition to an inner face of said substrate to form a composite material such that said composite material receives and retains the liquid when the liquid flows from a first planar dimension to a second planar dimension.

26. The process of claim 25, wherein said leakage prevention means is formed at the area of contact between said substrate and an edge of the said second applicator roll.

27. The process of claim 26, wherein said composite material forms an absorbent article and said leakage prevention means is positioned in said absorbent article to prevent leakage from the periphery of said absorbent article.

28. The process of claim 27, wherein said chemical treatment induces wetting of said substrate by a liquid.

29. The process of claim 28, wherein said chemical treatment comprises from about 1% to about 100% chemical treatment and from about 0% to about 99% water.

30. The process of claim 29, wherein said substrate is selected from the group comprising woven fabric, nonwoven fabric, foam, knitted fabric, and film.
